(19) United States
(12) Patent Application Publication
(10) Pub. No.: US 2010/0023094 A1
(43) Pub. Date: Jan. 28, 2010

(54) System for the Management of Arousal Levels of a Subject

(76) Inventors: Kathryn Leigh Smith, Cornwall (GB); Jeremy John Smith, Cornwall (GB)

Correspondence Address: Kimberly A. Chasteen, PO Box 1243, Yorktown, VA, 23692, US

(21) Appl. No.: 12/522,542

(22) PCT Filed: Jan. 8, 2008
(86) PCT No.: PCT/GB08/00041

§ 371 Date: Aug. 27, 2009

(30) Foreign Application Priority Data
Jan. 8, 2007 (GB) 0700227.2

(51) Int. Cl. A61N 1/00

(52) U.S. Cl. 607/62

(57) ABSTRACT

An interactive system (2) for providing sensory stimulation to a subject (30), the interactive system comprising a main (4) and satellite housings (14, 16), the housing being portable across surfaces, whilst still able to be secured to fixtures or other fittings while in use; a plurality of sensory stimulation means disposed within the main or satellite housings and adapted for providing sensory stimulation to the environment surrounding the housing, the sensory stimulation means providing stimulation for one or more smell, sound, wherein the system comprises interactive processor, remote control devices (34), switches and/or physiological measurement equipment (32). The sensory stimulation means may be electrical or non-electrical. The interactive system preferably comprises a power storage and delivery device, such as a battery. The housing is preferably arranged without sharp edges or protrusions, in order to avoid harm to users and is capable of being cleaned in line with current hospital clinical infection control procedures.

The present invention relates to the management of arousal levels of subjects and patients and to a system for performing the same. The invention relates in particular to a system for providing sensory treatment and aid to a subject or patient to manage fluctuating levels of alertness, distress and agitation.

There are many situations where a subject may have their level of arousal change. Patients suffering from psychological disorders may experience such changes as symptoms of their condition. Other subjects may experience similar changes in arousal state as a result of a wide range of situations, including factors arising in the work or home environment. For example, a person using a computer may undergo or experience marked changes in their state of arousal as a result of the work. For instance, person working at a computer may experience a reduction in their arousal level, due to carrying out a tedious task, or may experience a heightened arousal level, as a result of finding the task at hand frustrating. Such significant changes in the arousal level of the person can severely affect their performance at the task, cognitive decision making, and ability to learn, as well as having a medium and long term impact on their wellbeing.

There is a need for a system for combatting such changes in the arousal level of a subject and improving the wellbeing of the subject over the short, medium and long term.

According to the present invention, there is provided a system for managing the arousal level of a subject, the system comprising:

means for monitoring the arousal level of the subject; and

means for stimulating one or more senses of the subject, responsive to the means for monitoring the arousal level of the subject, whereby the sensory stimulation alters the arousal state of the subject.

Arousal is an individual's capacity for consciousness and awareness as a result of responsiveness to sensory stimulation or excitability. It helps maintain a homeostasis between the internal and external environment of the individual. Arousal is a state of mind influenced by the various hormones, enzymes neurotransmitters and other chemicals in the body. It is usually a reaction to the bio-psycho-social environment we function in. Thus given the levels of stress a person encounters, their arousals fluctuate accordingly to cope and keep their body in a state of dynamic equilibrium with the outside world. Individuals have peaks and valleys in their arousal levels throughout the day. However most manage to maintain an appropriate level of arousal by various coping strategies. These strategies help the person to interact effectively with the environment. People who have difficulty maintaining an appropriate level of arousal spend more time in a high or low level of arousal and may constantly seek or avoid input to try and regulate arousal. This then prevents them from functioning at an optimum level and could impact on their day to day activities.

In one embodiment, the present invention provides an interactive system for providing sensory stimulation to a subject, the interactive system comprising:

a housing, the housing being freely portable, adjustable and moveable across and between surfaces, with the means to be temporarily fixed to other static or portable fittings and fixtures;

a processor to control, manipulate and alter a plurality of sensory stimulation means disposed within the housing and adapted for providing sensory stimulation to the environment surrounding the housing, the sensory stimulation means providing stimulation for one or more of the senses of touch, sight, smell, sound, taste, proprioception or vestibular;

a remote control unit or units to interact with, control and manipulate the processor and therefore the sensory stimulation means, this may include an ability to measure, record and feedback biometric and physiological and neurophysiological measurements to the processor;

wherein all the interactive system is self contained or housed within the housing or within similar satellite housings but connected through electrical, wireless or other means.

The system of the present invention alters any environment in order to assist individuals or groups of people to achieve the arousal level required in order to promote optimal functioning for a particular task or activity and to improve the general wellbeing of the subject or subjects. This is done by stimulating the senses in order to promote the sensory processing and integration ability of each individual in order to enhance an individual's adaptive responses (behaviours).

The system of the present invention comprises means for monitoring and/or measuring the arousal level of the subject. Such means may be any suitable means for measuring one or more biometric, physiological or neurophysiological indicators of the arousal level of the subject. The means may monitor or measure the one or more indicators directly, by direct intervention in or on the subject, for example by being in touch with the skin of the subject. Indicators of the arousal state of the subject that may be monitored in this way are known in the art and will be known to the person skilled in the art. For example, the means may measure one or more of the heart rate, respiration rate, or body temperature of the subject. Alternatively, the means may monitor the subject by indirect means. Again, indirect methods and means of monitoring the arousal level of the subject are known in the art. Examples include measuring the response time of the subject to a task, measuring movement of the subject, speech patterns of the subject, and the like.

The means for monitoring the arousal level of the subject may be a stand alone means, for example directed at or in contact with the subject. For instance, a subject may be provided with a means to hold or have attached to them, while performing a task or undergoing a given activity. Alternatively, the means may be incorporated into an article, device or instrument in the environment of the subject or being used by the subject. For example, the means for monitoring the subject may be incorporated into the mouse or keyboard of a computer system being used by the subject. Still further, the means for monitoring the subject may comprise one or more cameras and/or movement sensors.

The system of the present invention further includes the means to provide sensory input to either increase (alert) or decrease (calm) or maintain arousal levels to allow an individual or group of individuals to achieve the ‘just right state’ of arousal for a particular activity.

The means for providing sensory input to the subject are responsive to the means for monitoring the arousal level of the subject. In a preferred embodiment, the system further comprises a processor, the processor arranged to receive information from the means for monitoring the arousal level of the subject, processing the said information, and controlling the means for stimulating one or more senses of the subject. The processor determines the appropriate stimulations to be provided to the subject, in response to the detected arousal levels. The processor may comprise one or more in-built or pre-programmed routines for making such determinations. Alternatively, or in addition thereto, the processor may be adapted to be operated or programmed to provide a specific set of stimuli in response to specific monitored states of the subject, as discussed in more detail hereinafter.

Examples of arousal states that may be achievable through use of this invention include:
Calm and alert arousal level: in order to play, work, study, think clearly, make accurate decisions and remember or recall knowledge
Hypoalert (under alert) arousal level: this is requisite state pre falling sleep and can help induce/promote sleep
Hyperalert (over alert) arousal level: This state is typically ‘unhelpful’ except when superhuman effort is required e.g. in survival states of fright where ‘flight’, ‘fight’ or ‘freeze’ responses are key to survival.

Changing the environment to change arousal levels is helpful where a change in arousal level will enhance or promote improved ability of the subject to function, participate in or execute a particular task or activity.

Examples include:
Reducing arousal levels prior to operation or invasive medical intervention, or during waking surgery i.e. patient awake with only localised anaesthesia e.g. spinal block during hip operation or pre-cannulation in children, those in maternity and palliative care, the elderly and those suffering from dementia;
Reducing arousal levels of those subject groups who typically have hyper (over) responsive sensory systems, for example survivors of trauma or abuse, children with autism/ADHD, for some with neurological changes or neurodevelopmental changes;
Increasing arousal levels of a subject where required for tasks demanding high levels of concentration or increased attention or where the individual may have a generally hypo (under) responsive sensory systems. Examples where this may be the case include those with neurological and neurodevelopmental changes including those with dementia type illnesses, developmental delays, genetic anomalies, sensory impairment and autism.

The system of the present invention has a general application to a wide range of subjects and to a wide range of situations. One application for the system is in the assistance and treatment of subjects or patients with psychological problems or disorders.

Currently well equipped specially designed Sensory Rooms are common in many hospital and care settings in order to be able to offer subjects alternative strategies to manage altered states of arousal, including distress and agitation, assisting them to learn and develop practical coping strategies, enhance learning ability and reduce the need for some invasive medical and physical interventions including sedating injections and Control and Restraint or Seclusion procedures. The system of the present invention offers an alternative strategy and technology for increasing self management of subjects with mental health and/or other difficulties who are distressed or angry, or engaging in self harm behaviours.

Sensory approach assessment accurately predicts how subjects will respond at a physiological and neuropsychological level to specific interventions. This allows subjects to choose activities they enjoy doing, while allowing therapists to then predict which interventions, including alternative therapies, will be effective at either raising or decreasing arousal levels, enabling subjects to experience rapid success, promoting continued engagement with this approach. ‘Just Right’ sensory environments can then be created when the subject is in crisis, according to the subject's needs. The system of the present invention may be programmed to respond to the particular needs of an individual and to provide a specific set of sensory stimulations to the subject, as determined necessary by the monitoring and measuring means. The programmed responses may be determined by the aforementioned sensory approach assessment. This assessment and information may be entered by service users, including patients, clients, carers and parents.

Allocating space to be dedicated for use as a Sensory Room is becoming more difficult as services try to be flexible and provide more varied therapy options. To address this growing need for flexibility, portable Sensory Kits have been developed; these are often heavy, big and bulky, requiring many trips across sites to move equipment between storage areas and different buildings. Traditional ‘Sensory Carts’—old hospital trolleys stacked with plastic tubs of Sensory equipment, the obligatory battery operated CD Player and portable light projector—are just not good enough and look ‘home grown’ and unprofessional. As Health and Safety gets more stringent, how, when and where these homemade carts can be safely transported and used, means often clients cannot have access to equipment, or sometimes even if provided, it just cannot be powered in a convenient and, more importantly, safe manner. This restricts and denies clients therapeutic opportunities to learn how to use sensory strategies when distressed by emotions or events instead using more destructive ways, which can include harm of self or others.

Accordingly, there is a need to enable clients with mental health difficulties to have access to the therapeutic advantages of much of the equipment from a typical Sensory Room without services having to meet the high initial and ongoing expenses involved in provision of dedicated Sensory Rooms. The resource will ensure this therapy approach can be provided flexibly in a variety of locations where because of needing dedicated space, this type of approach would previously not be possible within existing/current room provision egg 136 De-escalation rooms, A and E Departments and maternity units.

The system of the present invention may be arranged to allow the subject to adjust the responses of the system to changes in the arousal level of the subject. Alternatively, the system may be arranged to provide a fixed response to the changes in the subject, as may be pre-programmed into the system or programmed into the system from time to time. The system may comprise means allowing it to be programmed directly and/or remotely, for example via a network, by wireless connection, or the like.

Further, the system may comprise means for recording and storing data relating to the arousal levels and arousal management history of the subject for subsequent retrieval.

The system of the present invention, in one embodiment is a self contained system, that is it comprises all the means necessary to provide sensory stimulation to a subject to achieve the required objective of therapy, calming, or the like within a single or multiple housings. Thus, the system may comprise a main housing and one or more satellite housings, connected to the main housing by a suitable connection means, such as a wired or wireless connection.

The system of the present invention is preferably secure, that is the interactive system must be of a design and construction that reduces the risk of harm to a subject or other person to a minimum. In this respect, components that may be used as a weapon or a projectile are preferably secured within the housing in general, in particular when the system is not in use. Similarly, the exterior of the system is preferably of a design that minimises the risks of subjects using the system as a whole to harm themselves or others.

The system of the present invention is preferably portable, being freely moveable across and between surfaces. It may be adjustable and with the means to be temporarily fixed to other static or portable fittings and fixtures, for example between hospital bed and operating theatre table or from room to room within a given facility.

In this way, the sensory stimulation may be provided to subjects at their location. In addition, this avoids the need for having a dedicated room or space for providing such sensory treatment, as is currently the practice and requirement with existing technology. To render the system fully moveable, the housing is preferably fitted with clamps, clips, cramps and or castors or wheels of conventional design. The system is most preferably moveable through 360° in three dimensions, in order to allow it to be moved and positioned for maximum effect, and within restricted and confined spaces.

Where Health and Safety guidelines are very strict, especially hospital units and operating theatres, the system will be constructed to meet current Health and Safety guidelines. In particular, the system may be constructed to be easily disinfected and cleaned in line with clinical infection control measures and to avoid any sharp edges or protrusions, ligature points or damaging projectiles that may be used by an agitated or semi-conscious or severely disturbed patient, unintentionally or intentionally, resulting in harm to themselves or others.

The housing may preferably be provided with portions, such as doors, flaps or removable lids, which may be selectively opened to reveal the appropriate sensory stimulation means, while also allowing the sensory stimulation means to be protected and secured out of reach when not in use. The housing may be provided with handles or grabs, in order to ease moving and maneuvering the interactive system. If provided, the handles or grabs are removable or, most preferably, inset into the housing, so as not to protrude.

The housing is preferably provided with means for securing it in a closed position, for example a suitable lock or retaining device, such that the sensory stimulation means may be secured in an inaccessible manner, to protect and conceal equipment and in some cases avoid harming a subject when not in use.

In a particularly preferred embodiment, the housing is provided with one or more portions that open to reveal the sensory stimulation means and provide access thereto. In this way, the housing may be closed, in order to secure the components of the system and prevent them from being damaged or being used to cause damage to themselves or others by a semiconscious, agitated or distressed subject.

Sensory stimulation means included in the system can be customised to meet the needs of particular client group. The sensory stimulation means may be electrical or non-electrical. Suitable means are known in the art.

The sensory stimulation means may be selected from means for stimulating the senses of sight, sound, smell, touch, taste, proprioception and vestibular. Preferably, the interactive system comprises means for stimulating a plurality of the aforementioned senses. A plurality of different means may be provided for stimulating a single sense. In a preferred embodiment, the interactive system comprises means for stimulating all of the aforementioned senses. The means may be adapted to all operate at the same or, more preferably, to be selectively operated alone or in combination with one or more other means, in order to meet the requirements of the subject. Such an interactive provides the most versatile embodiment and has the greatest range of therapeutic applications. However, alternative embodiments may have means for stimulating only some of the senses. The system may comprise a single means or a plurality of means for stimulating a given sense. A single means may act as a stimulant for more than one sense.

To stimulate the visual sense of the subject, the interactive system may include a visual display means, such as an array of lights, for example a light emitting diode (LED) array, a plasma screen or liquid crystal display of known construction. The means for stimulating the visual senses may be provided in an existing structure or assembly, for example in portions of a work space or room, or within a device or apparatus being used by the subject, for example a computer monitor, keyboard or other device,

In one embodiment, such a means for stimulating the visual sense is preferably retained within a satellite housing and adapted to be exposed to the subject, either by extending from within the housing or by having a portion of the housing open to reveal the screen. Additional visual stimuli may be provided by one or more separate light assemblies. These assemblies may act to provide lighting effects to the main or satellite housing. In such a case, the main or satellite housing may comprise one or more transparent or opaque portions, through which a light display may be viewed.

Alternatively, or in addition thereto, the interactive system may comprise one or more assemblies for projecting light into the environment around the system.

Additional or alternative visual display means may be provided, for example, electrically operated candles and the like. In this way, the interactive system may be used to provide an appropriate environment around a subject for treatment, for example by providing a softly lit environment to calm a disturbed or agitated subject.

In a preferred embodiment, the main or satellite housing comprises one or a plurality of portions that may be opened, the visual stimuli being provided when the one or more portions of the housing are in the open position, the visual stimuli not being visible when the one or more portions of the housing are in the closed position. In this respect, the one or more portions of the housing may be provided with a transparent or opaque lining on its inner surface, with a suitable light assembly being located between the housing and the lining. A suitable light assembly for such use is an array of LEDs. The LEDs may be arranged in any suitable or desired pattern behind the lining, for example they may provide a lighting trim around the edge of the portion.

The visual stimuli may be adapted to be manipulated by a subject or a healthcare specialist. For example, the display screens may be of the touch-screen design, whereby subjects may interact with the display by touching the screen. Alternatively, the visual display may be adapted to be manipulated by a subject or other person by way of a remote control device or one of the other sensory stimulation means, for example the means for stimulating touch.

The visual stimuli may be provided in various formats. Such formats include calming displays, in which a visual display is adapted to provide a calming effect on a subject. In addition, the visual stimuli may be adapted to have one or more alerting formats, in which the subject or other person is alerted and caused to respond. Any or all of these formats of visual display may be linked to cooperate with other sensory stimulation means, for example the means for generating aural stimuli.

Aural stimuli may be provided by a suitable means for projecting sounds. The aural stimuli may be located in any suitable location with respect to the subject. In one embodiment, the aural stimuli are generated from the main or satellite housing and processor. In an alternative embodiment, the system is provided with means for providing aural stimuli directly to the subject, for example by means of headphones or earpieces.

Suitable means for creating and projecting a sound are known in the art and include conventional interactive system for playing music or sounds recorded or stored on conventional media, such as tape, compact disc, and computer readable memory. Other means that may be used to provide an aural display include sound emitting devices, such as shakers and the like. Such devices may require direct or remote operation by the subject or another person. Alternatively, the system is provided with means to drive and move sound emitting objects or devices, such as rattles, shakers and the like.

The system may also comprise means for stimulating the olfactory system of the subject. Means for producing scents and smells to treat and calm a subject are known in the art. Suitable means include known aromatherapy burners and the like. The means for stimulating the olfactory system of the subject may be located at any suitable location with respect to the subject, for example in a main or satellite housing of the system.

In addition, the system may comprise one or more means to stimulate or capture touch, proprioceptive or vestibular input. Again, suitable means for stimulating these senses (touch proprioceptive and vestibular) are known in the art. Such means may be disposed in any suitable position with respect to the subject in order to provide the requisite stimulation to the subject, for example within the main or satellite housing. Alternatively, the means to stimulate or capture touch, proprioceptive or vestibular input may be arranged on remote control devices and switches and includes PDA and other similar technology, including body movement sensors.

As noted hereinbefore, the means to stimulate the subject by touch may be arranged to activate or control other sensory stimulation means, for example the means for stimulating sight, aural or olfactory responses. Suitable means include a touch-activated screen and one or more pressure pads located on or within the system. In this way, the subject may be caused or required to interact with the system and the sensory stimuli.

The system is preferably provided with one or more control means for activation and control of the sensory stimulation means. This may be any suitable processor or computer, with associated memory, such as RAM, ROM and/or hard disk data storage. Suitable processors for use in the interactive system are known in the art. The processor may be provided with all the sensory stimulation routines pre-programmed. Alternatively, or in addition, the processor may be provided with means to receive updated or modified routines, for example by way of a portable data storage device, such as a disk or chip, by biofeedback or physiological or neurophysiological measurement technology or by connection to a network, such as a local area network (LAN) or the internet.

In a preferred embodiment, one or more of the sensory stimulation means is powered electrically. In such a case, the system may comprise an independent power supply, in particular in the case that the system is a stand-alone, independent device. Such a power supply, for example a battery, may be disposed within the housing in such a way that access by unauthorized persons or subjects is restricted. For example, the independent power supply may be housed within a dedicated compartment within the housing.

The power storage and supply means is preferably rechargeable. The system is preferably provided with suitable connections, for example an electrical socket, to allow recharging. Again, however, it is most preferred that such connections are not exposed on or project from the outer surface of the housing, in order to prevent harm coming to a subject or other person.

An onboard power supply means the interactive can be safely used anywhere, with no risks of loose cables or the necessity of multiple vacant electrical sockets in places appropriate to locate and use equipment safely. This ensures the system can be quickly moved and when necessary it can be removed from a room or be relocated to more appropriate settings. In the case of an interactive system for use with subjects or areas of lower risk, the power storage means may be dispensed with and a conventional cable connection to a local power supply grid relied upon.

The housing may be constructed of any suitable material, including metal, plastic and/or wood. One preferred material is wood, for example birch or other form of plywood. An alternative preferred material is plastic, in particular injection molded plastic. Plastic or wood housings have the advantage of providing improved stimulation to touch, compared with metal housings. The housing may be covered in part or wholly with a coating or covering material. For example, the housing may have a soft washable vinyl or other padded covering. The covering or outer surface of the housing may comprise part or all of the means for stimulating the sense of touch. The housing may be painted or varnished.

In a further aspect, the present invention provides a computer system incorporating the arousal level management system, that is a computer system comprising a system as hereinbefore described, a display having a screen and one or more control components for use by an operator using the computer system; the monitoring means being disposed in one or more of the control components, and the stimulation means being disposed in one or more of the display and the control components.

The computer system is preferably arranged to have the monitoring means measure the arousal level of the user as a result of the user touching the one or more control components.

Examples of applications and uses for the system of the present invention include:
Hotels and residential homes;
Classrooms and other educational facilities;
Meeting rooms and conference facilities;
Theatres and entertainment venues;
Spas, gyms, leisure centres and sports venues;
Transport facilities and vehicles, including buses, trains, cars, planes and boats;
Hospitals, hospices and clinics;
PCs and other computerized devices;
Offices and occupational health applications;
Residential locations;
Portable units, such as furniture and electrical appliances.

Alternatively, the system may be arranged as a personal device to be worn or held by the subject, for example worn as a wristband or the like.

Embodiments of the present invention will now be described, by way of example only, with reference to the accompanying drawings in which:

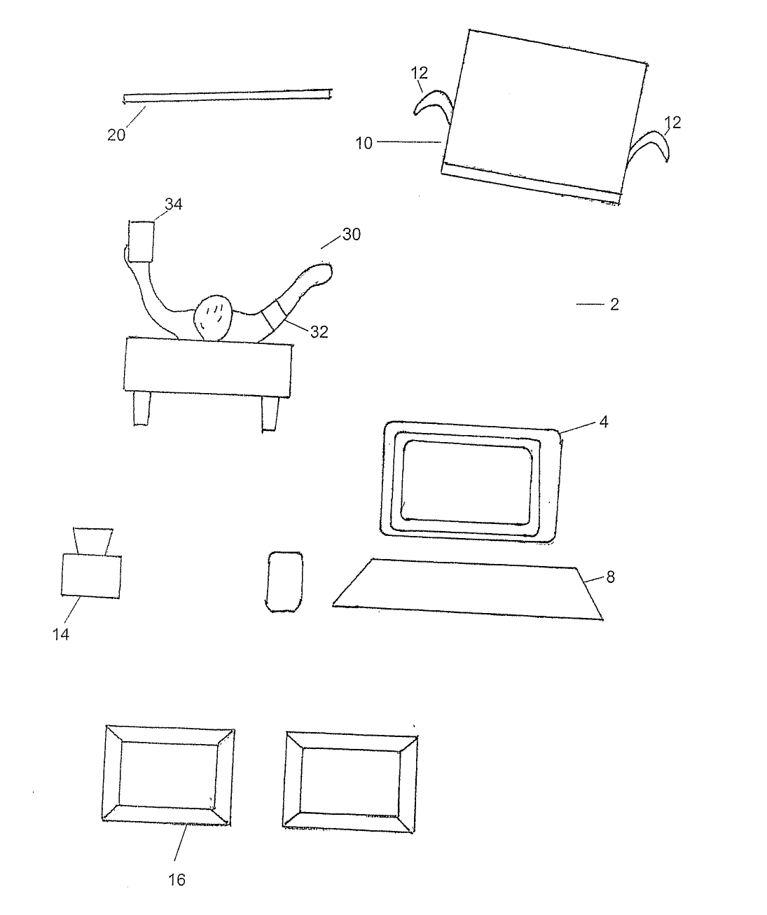

FIG. 1 is a perspective view of an embodiment of the system of the present invention;

Referring to FIG. 1, a system, generally indicated as 2 comprises a rectangular housing 4 that encases a processor and is freestanding or that can be clamped or cramped to a fixed or mobile surface or fixture. The housing 4 is provided with rounded edges and corners, and a smooth outer surface, in order to prevent injury or harm to a disturbed subject or other person.

The housing has a hinged front portion 8, which may be raised to open the housing to access the interior, as shown in FIG. 1. The housing may further comprise a display screen and other sensory stimulation equipment, for example a speaker and vapouriser.

A compartment may be provided in one end of the housing 4 to house a battery (not shown for clarity). A socket is provided in the end of the housing near the compartment to provide a connection for charging the battery by means of a conventional cable and plug assembly, which may be stored separately to the housing when not in use. The socket is flush with the outer surface of the housing.

As shown in FIG. 1, the system 2 further comprises a satellite unit 10, having a housing provided with handles 12 at one end. The handle shown in FIG. 1 extends from the housing. The handle is preferably retractable within the housing. An alternative and preferred embodiment provides a handle or grab assembly that is recessed into the housing, again to avoid harming a subject or other person. In a further alternative, the handle or grab may be omitted.

As shown in FIG. 1, when closed the overall approximate preferred dimensions of the interactive system are typically 380 mm long, 50 mm high and 250 mm deep. This will allow it to be easily moved between rooms or surfaces. The interactive system may be sized according to the duty to be performed and the needs of the particular facility or environment in which it will be used.

Referring to FIG. 1 the satellite unit 10 comprises a visual display screen, light projector and a speaker, but may also include vaporizer and other sensory stimulation equipment, as required by the setting. The visual display unit (VDU) and speakers may or may not be housed separately in further satellite housings. This is to facilitate maximum flexibility of use in small multifunctional spaces like operating theatres or high dependency units where the system's ease of movement is required in order to allow crucial theatre and other medical equipment or interactive system to be placed as required for a particular medical intervention, the system adapting around this.

Electrical equipment (which can be customized according to a particular unit or client groups needs) can be plugged into the battery and will then be mounted within, on or around the housing 4, either permanently or just while in use. Thus, a light projector 14 is shown which can be mounted to the upper surface of the housing at one end, which may be operated to provide specific lighting effects in the room or environment of the interactive system. Similarly, an aromatherapy assembly 16 for providing olfactory stimulation in the room or environment is shown. The assembly 16 may mounted within the housing 4 and exposed for use when the front portion is in the raised or open position.

As noted, the housing 4 comprises a lid portion 8, moveable about hinges, so as to open the entire top portion of the housing. In a preferred larger embodiment a pair of front portions may be pivoted about hinges and may be opened to expose the entire front of the interior of the housing. As also noted, the housing may be provided with cut out handles and clamps or cramps on either side to allow the interactive system to be pushed or pulled through 360 degrees for ease of positioning and to allow the system housing to be secured to fixed or portable surfaces and fixtures common in hospital, workplace, residential, educational and other settings.

Referring to FIG. 1, the sensory stimulation assemblies of the interactive system and their arrangement will now be described. Referring to FIG. 1, a visual stimulation means comprise several separate satellite components. First, an LCD screen 20 is mounted to a suitable surface. The screen may be used to display still or moving images, including videos and DVD's. The operation of the screen is controlled by a keyboard of conventional design, a PDA, a switch remote control system or by the processor and specialist interactive software responding to incoming physiological data according to preprogrammed instruction. The system may comprise suitable data storage means for the images to be displayed in the screen such as computer readable memory, compact disk (QD), digital video disk (DVD), tape or the like.

Second, light assemblies may be disposed on the interior surfaces of the housing 10 or satellite housing together with interior surfaces that are exposed and visible when the housing is opened. The light assemblies may be mounted behind an opaque or transparent lining that comprises the interior and exterior housing surfaces. The light assemblies may comprise strips of LEDs. Other suitable forms of light assemblies may also be employed.

Further, fibre optic cables may be provided to project light patterns from within the interactive system, for example located in a projection assembly mounted in the main or satellite housing.

Aural stimulation is provided by one or more speakers 22, which may be mounted to the main or satellite housing, or separate therefrom, as shown. The speakers may be controlled using the keyboard, a PDA, a switch remote control system or by the processor and specialist interactive software responding to incoming physiological data according to preprogrammed instruction. Again, suitable data storage means are provided to store sound data, as hereinbefore described, and including MP3 storage and playback means.

Other sensory stimulation means may be provided, as required by the duty to be performed. In particular, olfactory stimulation means, for example aromatherapy vaporizer assemblies, may be provided. These may be mounted in the main or satellite housing. Similarly, means for stimulating the vestibular, taste and/or proprioception senses may be provided.

Touch sense stimulation means may also be provided, again mounted in the main or satellite housing, or within a, PDA, a switch remote control system or by the processor and specialist interactive software and equipment responding to incoming physiological data according to preprogrammed instruction. This may include the means to provide massage or vibration stimulation. In a preferred embodiment the olfactory stimulation means and the touch sense stimulation means may be used as an interactive therapy tool for use with subjects.

As shown in FIG. 1, a subject 30 is provided with means for monitoring and measuring their arousal level. As shown, the subject 30 is fitted with a pad 32 in contact with the skin of the subject to measure the galvanic skin response of the subject. Data collected by the pad 32 is provided to the processor within the housing 4 and is used to control the various sensory stimulation means shown in the figure and described above. Transmission of data to and from the processor is by a wireless connection.

The subject 30 is provided with a control panel 34, with which to control the operation of the system 2. The control panel 34 may be omitted, allowing the system 2 to operate as an independent system. Alternatively, the control panel 34 may be operated by another person.

The subject 30 is shown in the figures as being in a prone position, as they may be when undergoing a medical procedure or treatment. It will be appreciated that the subject may be in any other suitable position and engaged in a wide range of other tasks, while having their arousal level monitored and controlled by the system, in order to provide the subject with the optimum level of mental and physical comfort for the task or situation in hand.

1. A system for managing the arousal level of a subject, the system comprising:
monitoring means for monitoring the arousal level of the subject; and
stimulation means for stimulating one or more senses of the subject, responsive to the means for monitoring the arousal level of the subject, whereby the sensory stimulation alters the arousal state of the subject.

2. The system according to claim 1, wherein the monitoring means operates by direct intervention on the skin of the subject or in the subject.

3. The system according to claim 1, wherein the monitoring means operate indirectly.

4. The system according to claim 1, wherein the monitoring measures one or more senses of the subject.

5. The system according to claim 1, further comprising a processor for receiving data from the monitoring means controlling the stimulation means.

6. The system according to claim 1, wherein the system is adapted to be operated and/or programmed remotely.

7. The system according to claim 1, further comprising means for recording data relating to the arousal levels and arousal management history of the subject.

8. The system according to claim 1, wherein the system is a self-contained unit comprising one or more housings.

9. The system according to claim 8, further comprising an energy storage means for providing power to the system.

10. The system according to claim 1, wherein the stimulation means are operable to stimulate one or more of the senses of sight, touch, sound, smell, taste, proprioception and vestibular of the subject.

11. The system according to claim 1, wherein the stimulation means are operable to stimulate more than one sense simultaneously.

12. The system according to claim 1, wherein a plurality of stimulation means are provided for stimulating a given sense.

13. A system for managing the arousal level of a subject, the system comprising:
a main housing, the housing being freely portable, adjustable and moveable across and between surfaces, with the means to be temporarily fixed to other static or portable fittings and fixtures;
a processor contained within the housing that can be programmed to respond to incoming data by software to control any or all of the sensory stimulation means;
a plurality of sensory stimulation means disposed within the main and/or one or more satellite housings and adapted for providing sensory stimulation to the individual and environment surrounding the housing, the sensory stimulation means providing stimulation for one or more of the senses of touch, sight, smell, sound, taste, proprioception or vestibular;
a remote control unit or switch unit to interact with or control and manipulate the processor and therefore the sensory stimulation means;
physiological and or neurophysiological measurement tools to measure, record and feedback biometric, physiological and or neurophysiological measurements to the processor wherein all the interactive system is self contained and housed within the main housing and/or within similar satellite housings but connected through electrical, wireless or other means.

14. The system according to claim 13, wherein the main or satellite housing is portable.

15. The system according to claim 13, provided with clamps, cramps or means to securely fix the housing to fixed or portable fixtures and fittings.

16. The system according to claim 13, wherein the main or satellite housing is provided with handles or grabs.

17. The system according to claim 16, wherein the handles or grabs are recessed into the housing, so as not to protrude from the plane of the surface of the housing, or are removable.

18. The system according to claim 13, wherein the housing is provided with a portion that may be opened or removed to access the interior of the housing.

19. The system according to claim 18, wherein the said portion forms part or all of the top of the housing.

20. The system according to claim 13, wherein the sensory stimulation means comprises a means for stimulating the visual senses of a subject.

21. The system according to claim 20, wherein the said means comprises a visual display screen.

22. The system according to claim 21, wherein the visual display screen is a plasma screen or liquid crystal display.

23. The system according to claim 20, wherein the said means comprises a light assembly.

24. The system according to claim 23, wherein the light assembly is operable to light a portion of the housing.

25. The system according to claim 23, wherein the light assembly is operable to project light into the surrounding environment.

26. The system according to claim 13, wherein the sensory stimulation means comprises a means for stimulating the aural sense of a subject.

27. The system according to claim 26, wherein the said means comprises one or more speakers for projecting a sound.

28. The system according to claim 27, wherein the speaker is arranged to project sound through a portion of the main or satellite housing or is exposed by opening a portion of the housing.

29. The system according to claim 13, wherein the sensory stimulation means comprises means for stimulating the olfactory sense of a subject.

30. The system according to claim 29, wherein the said means is adapted to provide aromatherapy.

31. The system according to claim 13, wherein the sensory stimulation means comprises means to stimulate the sense of touch, vestibular or proprioception of a subject.

32. The system according to claim 13, comprising means for stimulating a plurality of senses, the means for stimulating a first sense being linked with the activation of the means for stimulating a second sense.

33. The system according to claim 13, wherein one or more of the means for stimulating the sense requires interaction with a subject or other person.

34. The system according to claim 13, adapted to be responsive to incoming physiological and other programme data via a software programme that is preprogrammed to respond to specific physiological variables according to predefined rules.

35. The system according to claim 13, comprising a means for storing and/or providing power to one or more of the sensory stimulation means.

36. The system according to claim 35, wherein the means for storing and providing power is a battery.

37. The system according to claim 35, wherein the means for storing and providing power is rechargeable.

38. (canceled)

39. (canceled)

40. (canceled)
